(19) United States
(12) Patent Application Publication
(10) Pub. No.: US 2025/0242028 A1
(43) Pub. Date: Jul. 31, 2025

(54) METAL BORIDE NANOPARTICLE FOR CANCER DIAGNOSIS AND THERAPEUTIC TREATMENT

(71) Applicant: National Tsing Hua University, Hsinchu City (TW)

(72) Inventor: Kuo Chu Hwang, Hsinchu City (TW)

(73) Assignee: National Tsing Hua University, Hsinchu City (TW)

(21) Appl. No.: 18/656,585

(22) Filed: May 6, 2024

(30) Foreign Application Priority Data
Jan. 31, 2024 (TW) 113103656

(51) Int. Cl. A61K 41/00; A61K 47/64; A61K 47/68; A61K 47/69

(52) U.S. Cl. CPC A61K 41/0095; A61K 47/64; A61K 47/6851; A61K 47/6901; A61K 47/6923

(57) ABSTRACT

A metal boride nanoparticle is provided, which has dual functions of tumor diagnosis and therapeutic treatment. A surface of the metal boride nanoparticle is modified with antibodies, bioprobes, or coated with a biological cell membrane, and the antibodies or the bioprobes have a specificity for binding to receptors on specific tumor cells.

### CROSS-REFERENCE TO RELATED APPLICATION

This application claims the priority benefit of Taiwan application serial no. 113103656, filed on Jan. 31, 2024. The entirety of the above-mentioned patent application is hereby incorporated by reference herein and made a part of this specification.

### BACKGROUND

### Technical Field

The disclosure relates to a nanoparticle, and in particular, to a metal boride nanoparticle.

### Description of Related Art

The theoretical principle of neutron capture therapy (NCT) for cancer treatment has been successfully demonstrated clinically. The use of boron-10 atoms and gadolinium-157 atoms has super strong absorption ability for neutrons. After the boron-10 atom absorbs a neutron, it becomes unstable boron-11, and then rapidly nuclear fission produces high-energy α-particles and ⁷Li-particles, which can kill nearby cancer cells within the range of 10 μm to 15 μm. After the gadolinium-157 atom absorbs a neutron, it becomes unstable gadolinium-158. The unstable gadolinium-158 releases γ-ray, which damages the protein and DNA structure of surrounding cancer cells within a long distance of several centimeters, thereby killing the cancer cells. The therapeutic effect of neutron capture therapy depends on the accumulation of boron-10 atoms in the tumor (must be greater than 20-35 μg ¹⁰B/gram tumor tissue) and the ¹⁰B ratio of the tumor to the blood (must be greater than 3.0). Effective gadolinium neutron capture therapy must accumulate more than 50-200 μg ¹⁵⁷Gd/g tumor tissue in the tumor to effectively kill cancer cells. So far, most neutron capture therapies use small molecules containing boron atoms (such as p-boronophenylanaline, BPA) as neutron capture reagents. Small molecule boron-containing drugs lack the ability to actively target tumors, and the ratio of boron atoms or the number of gadolinium-157 atoms contained is too small to accumulate in the tumor so as to reach the threshold for effective treatment. There is no successful case of combining effective boron neutron capture therapy and gadolinium neutron capture therapy at the same time. “Effective” here means that the number of boron atoms and gadolinium-157 atoms accumulated in the tumor tissue exceeds the above-mentioned threshold values at the same time. Therefore, there are shortcomings of unexpected efficacy and many side effects.

On the other hand, regarding the use of near-infrared light photodynamic therapy and near-infrared light photothermal therapy to treat tumors and cancers, conventional technologies mostly use inorganic nanoparticles as photosensitizers to absorb near-infrared light to generate reactive oxygen species (ROS) or heat energy, so as to kill tumor cells. The wavelength of near-infrared light used is usually between 800 nm and 1550 nm. Since the wavelength of near-infrared light used in the existing technology is not long enough, the penetration depth into biological tissues is limited, resulting in the inability to treat deep-seated tumors or poor treatment results.

Based on the above, developing a metal boride nanoparticle, having the function of diagnosing and treating tumors, which can effectively accumulate a sufficient amount of boron-10 atoms in tumor tissue, and can absorb long wavelength infrared light that can effectively penetrate biological tissue (wavelength between 2000 nm and 7000 nm), so as to generate reactive oxygen species ROS or heat energy to kill tumor cancer cells, and has the ability to actively target specific tumors or tissues, is an important topic currently required for research . . .

### SUMMARY

The disclosure provides a metal boride nanoparticle, which has functions of tumor diagnosis and therapeutic treatment, so as to effectively accumulate a sufficient amount of boron-10 atoms and gadolinium-157 atoms in tumor tissue, absorb long wavelength infrared light that can effectively penetrate biological tissue (wavelength between 2000 nm and 7000 nm) to generate reactive oxygen species ROS or heat energy to kill tumor cancer cells, and has the ability to actively target specific tumors or tissues.

A metal boride nanoparticle is provided, which has functions of tumor diagnosis and therapeutic treatment. A surface of the metal boride nanoparticle is modified with antibodies, bioprobes, or coated with a biological cell membrane, the antibodies or bioprobes have the specificity to bind to receptors on the cell membrane surface of specific tumor cells, and therefore has the ability to actively target specific tumors or tissues.

In an embodiment of the disclosure, the biological cell membrane includes a red blood cell membrane.

In an embodiment of the disclosure, the metal boride nanoparticle binds to the receptors on the cell membrane surface of the tumor cells through the antibodies or the bioprobes so as to actively target tumor tissues.

In an embodiment of the disclosure, the receptors include folate receptors, fibroblast-activation protein receptors FAPI, epidermal growth factor receptors EGFR, or vascular endothelial growth factor receptors VEGFR.

In an embodiment of the disclosure, the tumor includes but not limited to melanoma, brain tumor, lung tumor, or head and neck tumor.

In an embodiment of the disclosure, after the metal boride nanoparticle is irradiated with a neutron beam, high-energy α-particles and ⁷Li particles, gamma rays or a combination thereof are produced.

In an embodiment of the disclosure, wherein the metal boride nanoparticle generates reactive oxides (ROS), heat energy or a combination thereof after irradiated a near-infrared light or a mid-infrared light, so as to kill tumor and cancer cells, and a wavelength 2 of an irradiation light is between 2000 nm and 7000 nm.

In an embodiment of the disclosure, the metal boride nanoparticle is doped with metal elements which emit near-infrared photoluminescence, the metal boride nanoparticle emits light in a range of 1100 nm to 2000 nm in a near-infrared region after being excited by irradiation with a near-infrared light, and a wavelength of irradiation with the near-infrared light is 800 nm to 1100 nm.

In an embodiment of the disclosure, the metal elements include Gd, Eu, Er, Ho, Yb, Pr, Cu, Cr, Ni or a combination thereof.

In an embodiment of the disclosure, based on a total moles of metal in the metal boride nanoparticle, a molar content ratio of the metal elements ranges from 0% to 100%.

In an embodiment of the disclosure, the near-infrared photoluminescence emitted by the metal boride nanoparticles is used in near-infrared photoluminescence imaging as a tool for diagnosing the location and size of tumors. In addition, magnetic metal boride nanoparticles can also be used as contrast reagents for nuclear magnetic resonance imaging (MRI); and because metal boride nanoparticles contain high atomic order metal elements, they can also be used as contrast reagents for computer tomography (CT).

In an embodiment of the disclosure, a particle size of the metal boride nanoparticle ranges from 5 nm to 300 nm.

In an embodiment of the disclosure, the metal boride nanoparticle (¹⁰B %≥20%) is prepared by microwave arcing method.

In an embodiment of the disclosure, based on a molar ratio of boron element in the metal boride nanoparticle, a content of isotope boron-10 is 20% to 100%.

In an embodiment of the disclosure, the antibodies include but limited to anti-EGFR antibodies, anti-VEGF antibodies, or TCR-like antibodies.

In an embodiment of the disclosure, the bioprobes include but not limited to bioprobes based on folic acid, TAT peptide, tumor fibroblast activation protein inhibitor (FAPI), RGD peptide or heparin (HEP) glycan.

Based on the above, the disclosure provides a metal boride nanoparticle, which has the function of diagnosing and treating tumors. By modifying the surface with antibodies, bioprobes or being coated with biological cell membranes, it can serve as a neutron capturing reagent with an active tumor target function. The metal boride nanoparticles of the disclosure can also be used as photosensitizers for near-infrared light and/or mid-infrared light photodynamic therapy and photothermal therapy. They can generate ROS or heat energy after irradiating near-infrared light and/or mid-infrared light, so as to treat deep-seated tumors. On the other hand, the metal boride nanoparticles of the disclosure are doped with metal elements that emit photoluminescence in the near-infrared light region, and can emit long-wavelength near-infrared light (1100 nm-2000 nm) after being irradiated with near-infrared light (800 nm-1100 nm) photoluminescence, which can be applied to near-infrared photoluminescence as a tool for diagnosing the location and size of tumors. In addition, magnetic metal boride nanoparticles can also be used as contrast reagents for nuclear magnetic resonance imaging (MRI); and because metal boride nanoparticles contain high atomic number metal elements, they can also be used as computer tomography (CT) contrast reagents. The metal boride nanoparticles of the disclosure also have a high content of boron-10 atoms, which can effectively accumulate a sufficient amount of boron-10 atoms in tumor tissues, and have the ability to actively target specific tumors or tissues.

### DESCRIPTION OF THE EMBODIMENTS

Hereinafter, embodiments of the disclosure will be described in detail. However, these embodiments are illustrative, and the disclosure is not limited thereto.

In the present specification, a range represented by “a numerical value to another numerical value” is a schematic representation for avoiding listing all of the numerical values in the range in the specification. Therefore, the recitation of a specific numerical range covers any numerical value in the numerical range and a smaller numerical range defined by any numerical value in the numerical range, as is the case with any numerical value and a smaller numerical range thereof in the specification.

The disclosure provides a metal boride nanoparticle, which has functions of tumor diagnosis and therapeutic treatment. A surface of the metal boride nanoparticle is modified with antibodies, bioprobes, or coated with a biological cell membrane, and the antibodies or the bioprobes have a specificity for binding to receptors on cell membrane surfaces of specific tumor cells.

In the present embodiment, the antibodies or bioprobes modified on the surface of metal boride nanoparticles have the specificity to bind to the cell membrane surface receptors of specific tumor cells. The antibodies may include, for example, anti-EGFR antibodies, anti-VEGF antibodies or TCR-like antibodies. The bioprobes may include, for example, bioprobes based on folate, TAT polypeptide, tumor fibroblast activation protein inhibitor (FAPI), RGD polypeptide, or heparin (HEP) polysaccharide. The receptors may include folate receptors, fibroblast-activation protein inhibitor FAPI, epidermal growth factor receptors EGFR, or vascular endothelial growth factor receptors VEGFR. Other antibodies or bioprobes that can bind to receptors of different tumor cells can also be used to modify the surface of metal boride nanoparticles. Since the surface-modified antibodies or bioprobes of metal boride nanoparticles have the specificity to bind to the receptors of specific tumor cells, the metal boride nanoparticles can bind to the receptors of the specific tumor cells through the antibodies or the bioprobes, so as to actively target tumor tissues. Therefore, the metal boride nanoparticles can be used as neutron capture reagents with active tumor targeting function or as photosensitizers for near-infrared and mid-infrared photodynamic therapy/photothermal therapy. The tumor cells or tumor tissues mentioned here include but are not limited to melanoma, brain tumors, lung tumors or head and neck tumors. The metal boride nanoparticles can bind to receptors of other different types of tumor cells based on different antibodies or bioprobes modified on the surface of metal boride nanoparticles, so as to actively target other different types of tumor tissues.

In this embodiment, a method for modifying antibodies or bioprobes on the surface of metal boride nanoparticles is, for example, using N,N′-dicyclohexylcarbodiimide (DCC for short) to couple carbonic acid groups and amino groups into amide group. As shown in the following formula (1), the metal boride nanoparticles are first coated with a polymer containing carbonate, and then DCC is used to couple the carbonate group on the polymer with the amino group on the antibody or bioprobes, or the metal boride nanoparticles are coated with amino-containing polymers, and then DCC is used to couple the amino groups on the polymer with the carbonate groups on the antibody or bioprobes.

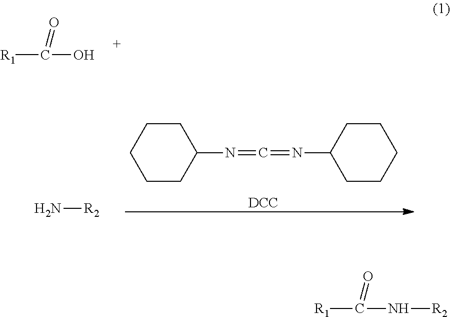

Here, R₁ and R₂ represent alkyl chains with different arbitrary chemical structures.

In this embodiment, the metal boride nanoparticles can also be coated by biological cell membranes, and the biological cell membranes can include red blood cell membranes. Because metal boride nanoparticles are coated with biological cell membranes, they can be used as neutron capture reagents with active tumor targeting functions or as photosensitizers for near-infrared and mid-infrared photodynamic therapy. The method of coating metal boride nanoparticles with biological cell membranes is described below, taking red blood cell membranes as an example, but the invention is not limited thereto. Membrane vesicles are first derived from red blood cells and then fused to the surface of metal boride nanoparticles. In more detail, purified red blood cells are treated with hypotonicity to remove their intracellular contents. Afterwards, the resulting red blood cell ghost is extruded through the porous membrane to generate red blood cell membrane-derived vesicles. The mechanical force provided by the extrusion process can promote the fusion of red blood cell membrane vesicles and metal boride nanoparticles, so that the red blood cell membrane can coat the metal boride nanoparticles.

In this embodiment, the metal boride nanoparticles can generate reactive oxygen species (ROS) after being irradiated with near-infrared light and/or mid-infrared light, and the irradiation wavelength λ is between 2000 nm and 7000 nm. In more detail, metal boride nanoparticles not only have the active tumor targeting function mentioned above, but can also be used as the photosensitizer of near infrared and/or mid-infrared photodynamic therapy and near infrared or mid-infrared photothermal therapy, so as to generate ROS or heat energy to kill cancer cells and treat deep-seated tumor cancers. In this way, the problem of conventional photodynamic therapy and photothermal therapy lacking photosensitizers that can absorb long-wavelength near-infrared light and mid-infrared light to generate active oxygen-containing free radicals and heat energy can be solved.

In this embodiment, the metal boride nanoparticles are doped with metal elements that emit photoluminescence in the near-infrared light region. After being excited by irradiation with near-infrared light (800 nm to 1100 nm), they can emit photoluminescence in the near-infrared range from 1100 nm to 2000 nm. By emitting light within the light zone, it can be used as a diagnostic tool for the presence of deep-seated tumors. The metal element may include but is not limited to Gd, Eu, Er, Ho, Yb, Pr, Cu, Cr or Ni. Based on the total mole of metal in the metal boride nanoparticles, the mole content ratio of the near-infrared light-emitting metal element ranges from 0% to 100%. 0% means that the metal boride nanoparticles do not contain metal elements that can emit photoluminescence, but only contain other metal elements that do not emit photoluminescence.

In this embodiment, the metal boride nanoparticles that can emit photoluminescence in the near-infrared light region can be used as a non-invasive fluorescent diagnostic tool, and the metal boride nanoparticles that contain magnetic metal ions can be used as nuclear magnetic resonance imaging tools (MRI) contrast reagents, and all metal boride nanoparticles can be used as contrast reagents for computed tomography (CT).

In this embodiment, the particle size of the metal boride nanoparticles may range from 5 nm to 300 nm. The precursor of metal boride nanoparticles can include boric acid enriched in the isotope boron-10. The metal ion salts and boric acid powder enriched in the isotope boron-10 are uniformly ground, and then prepared by a microwave arcing method. The microwave arc generates a plasma atmosphere with a temperature exceeding 1000° C., which is used for the decomposition of organometallic precursors and the recombination of high-energy atoms and clusters. Based on the molar ratio of boron element in metal boride nanoparticles, the content of isotope boron-10 can reach between 20% and 100%. In this way, a very small nanoparticle volume can contain an extremely large amount of boron-10 atoms, which can effectively solve the problem that small molecule drugs in the conventional technology contain too few boron-10 atoms and are difficult to accumulate a sufficient amount of boron-10 atoms in tumor tissues.

In summary, the metal boride nanoparticles of the disclosure have dual functions and effects of diagnosing and treating tumors. The surface is modified with antibodies, bioprobes or coated with biological cell membranes. The antibodies or bioprobes have a specificity for binding to receptors on specific tumor cells. In addition, the metal boride nanoparticles enriched with boron-10 of the disclosure are prepared by a microwave arcing method, and the molar content of the isotope boron-10 accounts for more than 20% of the total boron element. In this way, the metal boride nanoparticles of the disclosure can be used as neutron capture reagents with active tumor targeting functions, and can effectively accumulate a sufficient amount of boron-10 atoms in tumor tissues. On the other hand, the metal boride nanoparticles of the disclosure are doped with metal elements that can emit photoluminescence in the near-infrared light region (1100 nm to 2000 nm). After being excited by irradiation with near-infrared light (800 nm to 1100 nm), it can emit light in the near-infrared region of 1100 nm to 2000 nm, which can be used as a diagnostic tool for the presence of deep-seated tumors. It can also generate active oxygen free radicals (ROS) or heat energy after irradiation with mid-infrared light (2000 nm to 7000 nm) to kill tumor cancer cells. In addition, metal boride nanoparticles containing magnetic metal ions can be used as contrast reagents for nuclear magnetic resonance imaging (MRI), and all metal boride nanoparticles can be used as contrast reagents for computed tomography (CT). Therefore, metal boride nanoparticles of the disclosure can effectively solve the problem of traditional neutron capture therapy, which has the disadvantages of many side effects and the inability to use bioimaging technology to track the distribution of nanomedicine particles in the living body.

What is claimed is:

1. A metal boride nanoparticle, which has dual functions of tumor diagnosis and therapeutic treatment, wherein a surface of the metal boride nanoparticle is modified with antibodies, bioprobes, or coated with a biological cell membrane, and the antibodies or the bioprobes have a specificity for binding to receptors on cell membrane surfaces of tumor cells.

2. The metal boride nanoparticle according to claim 1, wherein the biological cell membrane includes a red blood cell membrane.

3. The metal boride nanoparticle according to claim 1, wherein the metal boride nanoparticle binds to the receptors of the tumor cells through the antibodies or the bioprobes so as to actively target tumor tissues.

4. The metal boride nanoparticle according to claim 3, wherein the receptors include folate receptors, fibroblast-activation protein inhibitor FAPI, epidermal growth factor receptors EGFR, or vascular endothelial growth factor receptors VEGFR.

5. The metal boride nanoparticle according to claim 1, wherein the tumor includes melanoma, brain tumor, lung tumor, or head and neck tumor.

6. The metal boride nanoparticle according to claim 1, wherein after the metal boride nanoparticle is irradiated with a thermal neutron beam, high-energy α-particles and ⁷Li particles, gamma rays or a combination thereof are produced.

7. The metal boride nanoparticle according to claim 1, wherein the metal boride nanoparticle generates reactive oxides (ROS), heat energy or a combination thereof after irradiated a near-infrared light or a mid-infrared light, and a wavelength λ of an irradiation light is between 2000 nm and 7000 nm.

8. The metal boride nanoparticle according to claim 1, wherein the metal boride nanoparticle is doped with metal elements which emit near-infrared photoluminescence, the metal boride nanoparticle emits light in a range of 1100 nm to 2000 nm in a near-infrared region after being excited by irradiation with a near-infrared light, and a wavelength of irradiation with the near-infrared light is 800 nm to 1100 nm.

9. The metal boride nanoparticle according to claim 8, wherein the metal elements include Gd, Eu, Er, Ho, Yb, Pr, Cu, Cr, Ni or a combination thereof.

10. The metal boride nanoparticle according to claim 8, wherein based on a total moles of metal in the metal boride nanoparticle, a molar content ratio of the metal elements ranges from 0% to 100%.

11. The metal boride nanoparticle according to claim 1, wherein a particle size of the metal boride nanoparticle ranges from 5 nm to 300 nm.

12. The metal boride nanoparticle according to claim 1, wherein the metal boride nanoparticle is prepared by microwave arcing method.

13. The metal boride nanoparticle according to claim 1, wherein based on a molar ratio of boron element in the metal boride nanoparticle, a content of isotope boron-10 is 20% to 100%.

14. The metal boride nanoparticle according to claim 1, wherein the antibodies include anti-EGFR antibodies, anti-VEGF antibodies, or TCR-like antibodies.

15. The metal boride nanoparticle according to claim 1, wherein the bioprobes include bioprobes based on folic acid, TAT peptide, tumor fibroblast activation protein inhibitor (FAPI), RGD peptide or heparin (HEP) glycan.
